(19) United States
(12) Patent Application Publication
(10) Pub. No.: US 2020/0170750 A1
(43) Pub. Date: Jun. 4, 2020

(54) SYSTEMS, DEVICES AND METHODS FOR SECURING CAMERAS AND SUPPLEMENTAL LIGHTING IN SURGICAL ENVIRONMENTS

(71) Applicant: Engineered Medical Solutions Co., LLC, Phillipsburg, NJ (US)

(72) Inventor: Daniel R. Coppersmith, Washington, NJ (US)

(21) Appl. No.: 16/203,779

(22) Filed: Nov. 29, 2018

(51) Int. Cl. A61B 90/00; F16M 13/02; F16C 11/06; G03B 17/56; F21V 21/29; A61B 90/30; A61B 90/50

(52) U.S. Cl. CPC A61B 90/361; F16M 13/022; F16C 11/0661; F16C 11/0695; A61B 2090/308; F21V 21/29; A61B 90/30; A61B 90/50; F16M 2200/06; G03B 17/561

(57) ABSTRACT

A system for use during surgical procedures includes a flexible arm having a plurality of ball and socket segments that are interconnected for pivoting relative to one another, a universal adapter coupled with a first end of the flexible arm, and a component adapter coupled with a second end of the flexible arm. The component adapter includes a ball component that forms a snap-fit connection with one of the ball and socket segments. The universal adapter includes a male projection having a free end, an outer surface, external threads provided on the outer surface, a locking flange, and an annular groove formed in the outer surface of the male projection that is located between the locking flange and the external threads. The system has an overhead light connector having a female opening with internal threads. The external threads of the male projection are threaded into the internal threads of the female opening for securing the universal adapter to the overhead light adapter. The overhead light connector has a locking assembly with a locked position that enables the universal adapter to be unthreaded from the overhead light connector and a locked position that prevents the universal adapter from being unthreaded from the overhead light connector.

### BACKGROUND OF THE INVENTION

### Field of the Invention

The present patent application is generally related to surgical procedures, and is more specifically related to systems, devices and methods for securing video cameras, still picture cameras and supplemental lighting within surgical environments.

### Description of the Related Art

There have been many efforts directed to recording surgical procedures using video cameras and still picture cameras. After the surgery has been completed, the captured videos and pictures may be reviewed and studied for educational purposes by physicians, surgical assistants, medical students, patients and the like. The recorded video clips and pictures can also be used for directing discussions of surgical skills among peers, and for practicing long-distance medicine via real-time data transmissions. Moreover, the recorded information can be used by insurance carriers and attorneys to evaluate liability issues and medical malpractice claims.

For example, U.S. Patent Application Publication No. 2009/0086495 to Chen discloses a surgical lighting system with an image-recording function including a surgical light set and a flexible tube-shaped video camera connected to the surgical light set. The surgical light set has a fixed base, a rotary arm set pivoted to the fixed base, the rotary arm set comprising at least one rotary arm, and a surgical light connected to one rotary arm of the rotary arm set.

U.S. Pat. No. 4,963,903 to Cane discloses a system for positioning a camera relative to a work area. The system has a clamp situated outside of the work area, and an extension and support element which operates to maintain a given position. The system includes a camera mechanically held within a socket at an end of a position retention element. A high resolution solid state and miniature video TV camera serves as the camera. The system provides close-up monitoring or video-taping in which the video camera can be positioned directly over the area of surgical operation or industrial inspection. The camera can be moved and rotated to any position offering extreme macro close-ups, which can be video-taped for later use in educational and quality control processes.

U.S. Pat. No. 6,290,645 to Goldfarb et al. discloses a system for photographing an open surgical procedure. The system includes a high resolution endoscopic video camera, a surgical lamp having a handle covered by a sterile, compressible sheath and being positioned in spaced relation to an open surgical site, and a clamp for securing the video camera to the handle.

There have also been many efforts directed to providing sufficient lighting within operating room environments to better illuminate surgical environments and improve lighting conditions.

For example, U.S. Patent Application Publication No. 2013/0113909 to Deland discloses a system for illuminating and recording an image of an operative field during a medical procedure. The system includes a first light source assembly, a second light source assembly, and a controller. The first and second light source assemblies include a light source operable to illuminate the operative field, a camera operable to record the image of the operative field, and a sensor operable to detect motion from the light source assemblies. The controller is in communication with the first and second light source assemblies, and is configured to send operational commands to the light source assemblies based on feedback from the sensors.

U.S. Pat. No. 5,803,905 to Allred et al. discloses a surgical camera assembly and a method for visually producing an operative field with enhanced focal illumination. A retaining assembly is operably disposed in connection with a light source. The retaining assembly provides a sterile surface for orienting the light source in relation to the operative field and includes a means for retaining a first end of a camera lens. Removably disposed in engagement with the retaining assembly, the camera lens has an elongated body and a variable focus and zoom capability for producing visual images of the operative field. The elongated body of the camera lens is removably introduced within a channel formed in the body of a housing assembly. The housing assembly provides a sterile surface for manually adjusting the variable focus and zoom capability of the camera lens for purposes of enhancing the visual images of the operative field produced by the lens. In operation, a camera head converts the visual images produced by the camera lens into electrical signals. The electrical signals are transmitted by an electrical cable to a remote camera controller. The camera controller processes the electrical signals received from the camera head and converts these electrical signals into visual images for display on a display screen. The visual images of operative procedures may be recorded.

In spite of the above advances, there is a continuing need for improved systems devices and methods for accurately, effectively, and efficiently recording surgical procedures within surgical environments. There is also a need for systems, devices and methods for securing video cameras, still picture cameras and supplemental lighting within surgical environments.

### SUMMARY OF THE INVENTION

In one embodiment, a system for use during surgical procedures preferably includes a flexible arm having a first end and a second end, the flexible arm including a plurality of ball and socket segments that are interconnected, whereby adjacent ones of the interconnected ball and socket segments are configured to pivot relative to one another. In one embodiment, the system desirably includes a universal adapter coupled with the first end of the flexible arm, and a component adapter coupled with the second end of the flexible arm. The component adapter desirably includes a ball component that forms a snap-fit connection with one of the ball and socket segments.

In one embodiment, the system preferably includes a camera secured to the component adapter. The interconnected ball and socket segments desirably enable the second end of the flexible arm to flex relative to the first end of the flexible arm for selectively modifying the orientation of the camera.

In one embodiment, the system includes a light fixture secured to the component adapter. The interconnected ball and socket segments desirably enable the second end of the flexible arm to flex relative to the first end of the flexible arm for selectively modifying the orientation of the light fixture.

In one embodiment, the universal adapter preferably includes a male projection having a free end, external threads provided on an outer surface of the male projection, a locking flange provided at the free end of the male projection, and an annular groove formed in the outer surface of the male projection that is located between the locking flange and the external threads.

In one embodiment, the system desirably includes an overhead light connector having a female opening with internal threads. In one embodiment, the external threads of the male projection are preferably threaded into the internal threads of the female opening of the overhead light connector for securing the universal adapter to the overhead light adapter.

In one embodiment, the overhead light connector preferably has an end coupled with an overhead light for securing the universal adapter and the flexible arm to the overhead light.

In one embodiment, the overhead light connector preferably includes a locking assembly that is moveable between a locked position that enables the universal adapter to be unthreaded from the overhead light connector and a locked position that prevents the universal adapter from being unthreaded from the overhead light connector.

In one embodiment, the locking assembly may include a locking ring that is seated in the annular groove of the male projection when the locking assembly is in the locked position, and that is spaced from the annular groove of the male projection when the locking assembly is in the unlocked position.

In one embodiment, the system may include a post mount adapter with a body having a first end, a second end, and a central conduit extending from the first end to the second end. The post mount adapter preferably includes a first female opening located at the first end of the body that is adapted to receive the male projection of the universal adapter. The first female opening preferably has internal threads that are adapted to mesh with the external threads of the male projection of the universal adapter. In one embodiment, the post mount adapter preferably includes a second female opening located at the second end of the body of the post mount adapter that is adapted to slide over a post. In one embodiment, the system desirably includes a post having an end disposed within the second female opening of the post mount adapter for securing the post mount adapter to the post.

In one embodiment, the post mount adapter desirably has a locking assembly that is moveable between a locked position that enables the universal adapter to be unthreaded from the post mount adapter and a locked position that prevents the universal adapter from being unthreaded from the post mount adapter.

In one embodiment, the locking assembly of the post mount adapter desirably includes a locking ring that is seated in an annular groove of the male projection when the locking assembly is in the locked position, and that is spaced from the annular groove of the locking assembly when the locking assembly is in the unlocked position.

In one embodiment, the system preferably has a mounting base having a first end that is connected with the universal adapter, a second end having a socket component that forms a snap-fit connection with one of the ball and socket segments of the flexible arm, and an elongated handle that extends between the first end and the second end of the mounting base.

In one embodiment, a system for use during surgical procedures preferably includes a flexible arm having a first end and a second end, the flexible arm including a plurality of ball and socket segments that are interconnected, whereby adjacent ones of the interconnected ball and socket segments are configured to pivot relative to one another.

In one embodiment, the system may include a universal adapter coupled with the first end of the flexible arm. The universal adapter preferably includes a male projection having a free end and an outer surface that extends to the free end, external threads provided on the outer surface of the male projection, a locking flange provided at the free end of the male projection, and an annular groove formed in the outer surface of the male projection that is located between the locking flange and the external threads.

In one embodiment, the system desirably includes an overhead light connector having a female opening with internal threads. In one embodiment, the external threads of the male projection of the universal adapter are threaded into the internal threads of the female opening of the overhead light connector for securing the universal adapter and the flexible arm to the overhead light connector. In one embodiment, the overhead light connector preferably includes a locking assembly that is moveable between a locked position that enables the universal adapter to be unthreaded from the overhead light connector and a locked position that prevents the universal adapter from being unthreaded from the overhead light connector.

In one embodiment, the system may include a camera adapter coupled with the second end of the flexible arm. The camera adapter desirably includes a ball component that forms a snap-fit connection with one of the ball and socket segments.

In one embodiment, a camera is preferably secured to the camera adapter. In one embodiment, the interconnected ball and socket segments preferably enable the second end of the flexible arm to flex relative to the first end of the flexible arm for selectively modifying the orientation of the camera.

In one embodiment, the camera may be an electronic camera, a digital camera, a video camera, a still camera, and/or a film camera. In one embodiment, the camera may be in wireless communication with a central processing unit for transmitting captured images (e.g., video, still pictures) to the central processing unit. The captured images may be stored in a memory device or in the Cloud.

In one embodiment, a kit for recording surgical procedures and providing supplemental lighting in a surgical environment may include a plurality of ball and socket segments that are configured to be interconnected for forming a flexible arm. In one embodiment, each ball and socket segment may include a ball component at a first end and a socket component at a second end. In one embodiment, one or more ball and socket segments may be added to the flexible arm for increasing a length of the flexible arm or removed from the flexible arm for reducing the length of the flexible arm. The flexible arm may be modular so that its length may be modified based upon the needs of surgical personnel within a surgical environment.

In one embodiment, a kit may include a universal adapter securable to at least one of the ball and socket segments.

In one embodiment, a kit may include a component adapter having a ball component configured to form a snap-fit connection with at least one of the ball and socket segments.

In one embodiment, a kit may include a camera securable to the component adapter.

In one embodiment, a kit may include a light fixture securable to the component adapter.

In one embodiment, a kit may include an overhead light connector having a female opening with internal threads. In one embodiment, the universal adapter preferably has external threads on a male projection thereof that are adapted to be threaded into the internal threads of the female opening of the overhead light connector for securing the universal adapter to the overhead light adapter.

In one embodiment, a kit may include a post mount adapter including a body having a first end, a second end, and a central conduit extending from the first end to the second end. In one embodiment, the post mount adapter may include a first female opening located at the first end of the body that is adapted to receive the male projection of the universal adapter. In one embodiment, the first female opening preferably has internal threads that are adapted to mesh with the external threads of the male projection of the universal adapter. In one embodiment, the post mount adapter has a second female opening located at the second end of the body of the post mount adapter that is adapted to slide over a post.

In one embodiment, the overhead light connector may include a locking assembly that is moveable between a locked position that enables the universal adapter to be unthreaded from the overhead light connector and a locked position that prevents the universal adapter from being unthreaded from the overhead light connector.

In one embodiment, the post mount adapter may include a locking assembly that is moveable between a locked position that enables the universal adapter to be unthreaded from the post mount adapter and a locked position that prevents the universal adapter from being unthreaded from the post mount adapter.

These and other preferred embodiments of the present invention will be described in more detail below.

### BRIEF DESCRIPTION OF THE DRAWINGS

FIG. 1 shows a perspective view of a system for securing a camera to an overhead light, the system including a camera adapter, a flexible arm having ball and socket segments, a base, a universal adapter, and an overhead light connector, in accordance with one embodiment of the present patent application.

FIG. 2 shows a magnified view of a camera, the camera adapter and the flexible arm shown in FIG. 1.

FIG. 3 shows a magnified view of a proximal end of the flexible arm, the base, the universal adapter, and the overhead light connector shown in FIG. 1.

FIG. 4A shows a front elevation view of a ball and socket segment of the flexible arm shown in FIG. 1.

FIG. 4B shows a perspective view of the ball and socket segment shown in FIG. 4A.

FIG. 5A show a top perspective view of the camera adapter shown in FIG. 1.

FIG. 5B shows a bottom perspective view of the camera adapter shown in FIG. 1.

FIG. 6 shows a front elevation view of a system for securing a camera to an overhead light, the system including a flexible arm having ball and socket segments that are snap-fit together, in accordance with one embodiment of the present patent application.

FIG. 7 shows a front elevation view of a system for securing a camera to an overhead light having a flexible arm that is shorter than the flexible arm shown in FIG. 6.

FIG. 8A shows a method of securing the system of FIG. 1 to an overhead light, in accordance with one embodiment of the present patent application.

FIG. 8B shows the system of FIG. 8A after it has been secured to the overhead light of FIG. 8A.

FIG. 8C shows the system of FIG. 8B with the flexible arm in a bent configuration.

FIG. 9 shows the universal adapter of FIG. 1 and a post mount adapter utilized for securing the system of FIG. 1 to a post, in accordance with one embodiment of the present patent application.

FIG. 10 shows a method of coupling the system of FIG. 1 with a post using the post mount adapter of FIG. 9, in accordance with one embodiment of the present patent application.

FIG. 11A shows a first step of a method of securing the system of FIG. 1 to the post mount adapter of FIG. 9, in accordance with one embodiment of the present patent application.

FIG. 11B shows the system of FIG. 11A after it has been secured to the post mount adapter of FIG. 9, in accordance with one embodiment of the present patent application.

FIG. 12A shows a top perspective view of a camera adapter for a system for securing a camera in a surgical environment, in accordance with one embodiment of the present patent application.

FIG. 12B shows a bottom perspective view of the camera adapter shown in FIG. 12A.

FIG. 13A shows a method of securing supplemental lighting to an overhead light, in accordance with one embodiment of the present patent application.

FIG. 13B shows a system for securing supplemental lighting to an overhead light, in accordance with one embodiment of the present patent application.

FIG. 14A shows a perspective view of a sanitary sleeve for a system for securing cameras and supplemental lighting in surgical environments, in accordance with one embodiment of the present patent application.

FIG. 14B shows a front elevation view of the sanitary sleeve shown in FIG. 14A.

FIG. 15 shows the sanitary sleeve of FIGS. 14A and 14B being secured over a system for securing supplemental lighting to an overhead light, in accordance with one embodiment of the present patent application.

FIG. 16 shows a perspective view of a system for securing supplemental lighting in a surgical environment, in accordance with one embodiment of the present patent application.

FIG. 17A shows a first step of a method of securing supplemental lighting to a post within a surgical environment, in accordance with one embodiment of the present patent application.

FIG. 17B shows a second step of a method of securing supplemental lighting to a post within a surgical environment.

FIG. 18 shows a system for providing supplemental lighting within a surgical environment, the system having a flexible arm in a bent configuration, in accordance with one embodiment of the present patent application.

### DETAILED DESCRIPTION OF PREFERRED EMBODIMENTS

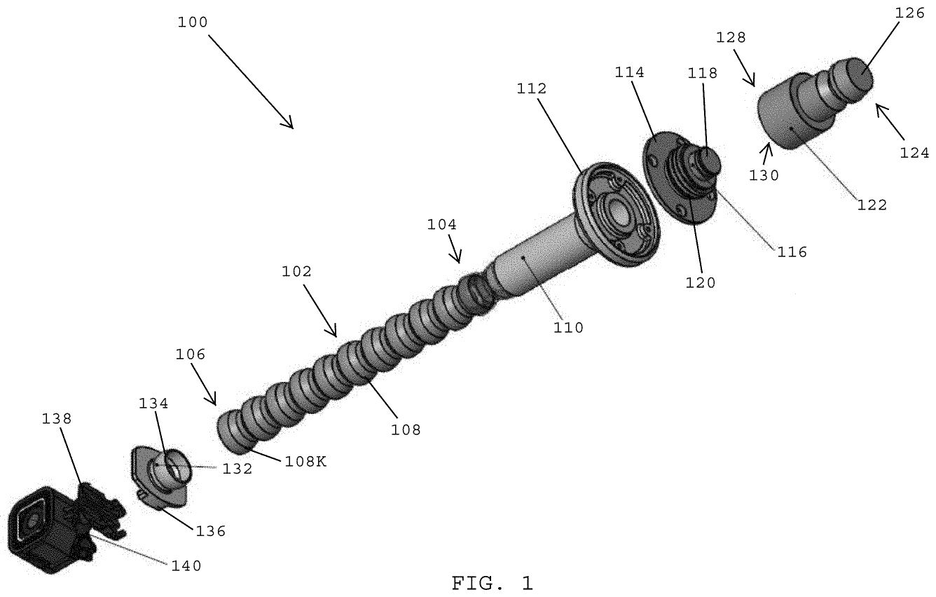

Referring to FIG. 1, in one embodiment, a system 100 for securing a camera to an overhead light preferably includes a flexible arm 102 having a proximal end 104 and a distal end 106. In one embodiment, the flexible arm 102 preferably includes a plurality of ball and socket segments 108 that may be snap-fit together to provide an articulating assembly that may be bent, flexed and/or twisted into a desired configuration and orientation. The overall length of the flexible arm 102 may be modified by changing the number of ball and socket segments 108 that are connected together. In one embodiment, a flexible arm may be selectively lengthened by attaching additional ball and socket segments to an existing arm. In one embodiment, a flexible arm may be selectively shortened by removing one or more ball and socket segments from an existing arm. In one embodiment, surgical personnel may desire to change the length of a flexible arm in response to the parameters of an operating room environment and the particular type of surgical procedure that is being performed.

In one embodiment, the segments 108 are interconnected to form a flexible assembly such as the flexible arm 102. In one embodiment, each segment 108 preferably includes a body with first and second end portions. An external socket engaging surface, such as a partially spherical or ball-shaped surface, may be provided at one end of the body. The other end of the body has an internal socket defining cavity bounded by an interior wall. The socket engaging surface of one segment is inserted and/or snapped into the socket defining cavity of another segment to interconnect the segments. In one embodiment, the ball and socket segments may have a longitudinal axis that extends between the first and second ends. In one embodiment, the segments 108 may be similar in construction to those sold under the trademark LOC-LINE by Lockwood Products Inc. of Lake Oswego, Oreg. In one embodiment, the segments may have motion limiters or stops to restrict the relative pivoting of adjacent segments for minimizing possible decoupling of the segments that could result from pivoting to extreme misaligned positions, as disclosed in U.S. Pat. Nos. 5,449,206 and 6,042,155 to Lockwood, the disclosures of which are hereby incorporated by reference herein.

In one embodiment, the system 100 preferably includes a mounting base 110 that is secured to the proximal end 104 of the flexible arm 102. The mounting base 110 may have a socket 111 located at a distal end thereof that is adapted to form a snap-fit connection with one of the ball and socket segments of the flexible arm. In one embodiment, the mounting base 110 desirably includes a mounting base flange 112 that opposes a universal adapter flange 114 of a universal adapter 116. In one embodiment, the universal adapter 116 desirably includes a male projection 118 having external threads 120.

In one embodiment, the system 100 desirably includes an overhead light connector 122 having a proximal end 124 with a male projection 126 and a distal end 128 with an internally threaded female opening 130 that is adapted to mesh with the external threads 120 on the male projection 118 of the universal adapter 116.

In one embodiment, the system 100 preferably includes a camera adapter 132 having a ball component 134 that is adapted to be snap-fit into an opposing female socket of a distal-most ball and socket segment 108K of the flexible arm 102. In one embodiment, the camera adapter 132 desirably includes a camera attachment flange 136 that is configured to engage an opposing attachment flange 138 on a camera 140 for securing the camera to the camera attachment flange.

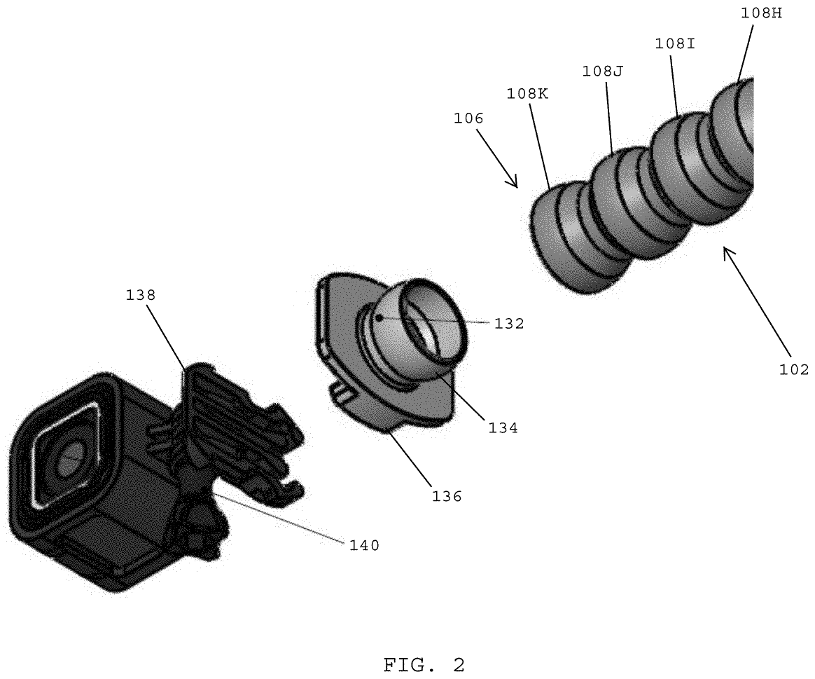

Referring to FIG. 2, in one embodiment, a distal end 106 of the flexible arm 102 (FIG. 1) desirably includes ball and socket segments 108H-108K that are snap-fit together whereby the individual ball and socket segments may be pivoted, flexed and rotated relative to one another for flexing, bending and/or twisting the flexible arm 102 to a desired configuration. In one embodiment, the ball component 134 of the camera adapter 132 may be snap-fit into the socket at the distal end of the distal-most ball and socket segment 108K. After the ball component 134 of the camera adapter 132 has formed a snap-fit connection with the distal-most ball and socket segment 108K, the camera adapter 132 may be pivoted and rotated relative to the ball and socket segment 108K.

In one embodiment, the camera 140 may be connected with the camera adapter 134 by interconnecting the attachment flange 138 of the camera 140 with the camera adapter attachment flange 136 of the camera adapter 132.

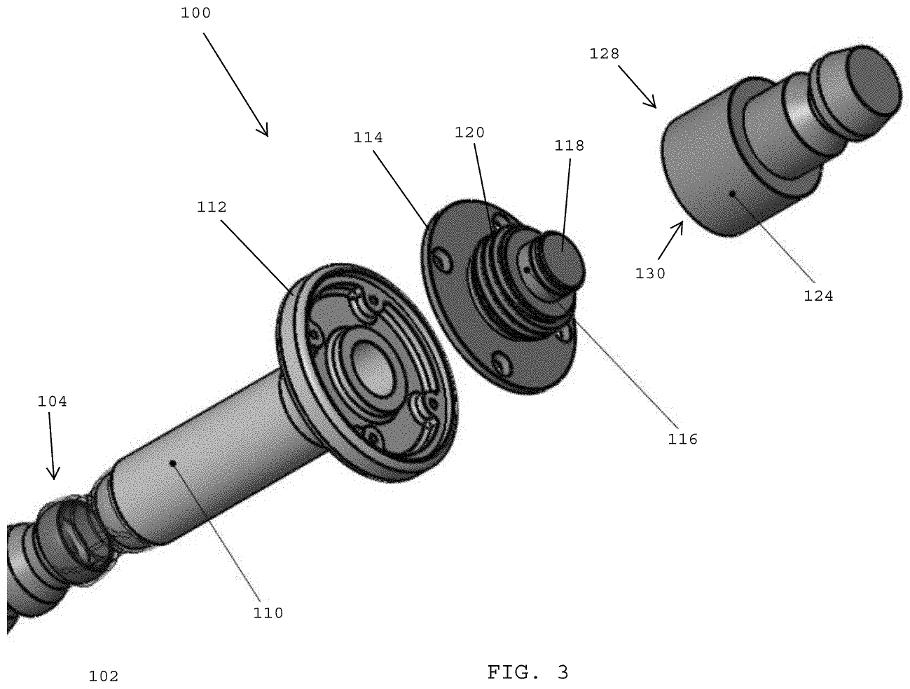

Referring to FIG. 3, in one embodiment, the proximal end of the system 100 shown in FIG. 1 preferably includes the proximal end 104 of the flexible arm 102 coupled with the mounting base 110. In turn, the mounting base flange 112 is adapted for being connected with the universal adapter flange 114 of the universal adapter 116. The external threads 120 on the male projection 118 of the universal adapter 116 are adapted to mesh with internal threads provided at the female end 128 of the overhead light connector 124.

In one embodiment, the overhead light connector 124 may include a locking assembly (e.g., an overheard light connector locking assembly) that prevents the universal adapter 116 from being inadvertently uncoupled from the overhead light connector 124. In one embodiment, when the locking assembly is in a locked position, the universal adapter 116 may not be unthreaded from the overhead light connector 124, thereby preventing inadvertently uncoupling of the universal adapter 116 from the overhead light connector 124. In one embodiment, the locking assembly may include a locking ring that is disposed inside the female opening 130 at the distal end of the overhead light connector 124. The locking assembly may include a spring that holds the locking ring in the locked position. In one embodiment, the locking assembly may include an actuator, such as a depressible button accessible on the outer surface of the overhead light connector 124, that moves the locking ring from the locked position to an unlocked position whereupon the universal adapter 116 may be unthreaded from the overhead light connector 124 for detaching the system 100 from an overhead light. In one embodiment, the shape, dimensions and configuration of the overhead light connector 124 may be modified to accommodate attachment to different types of connecting structure found on overhead lights.

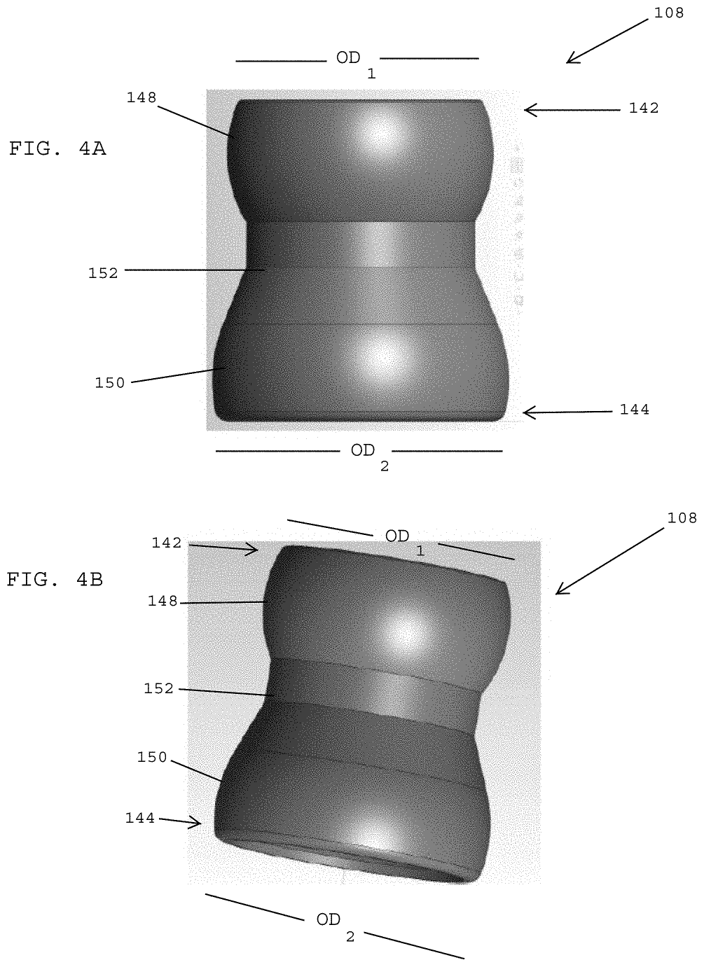

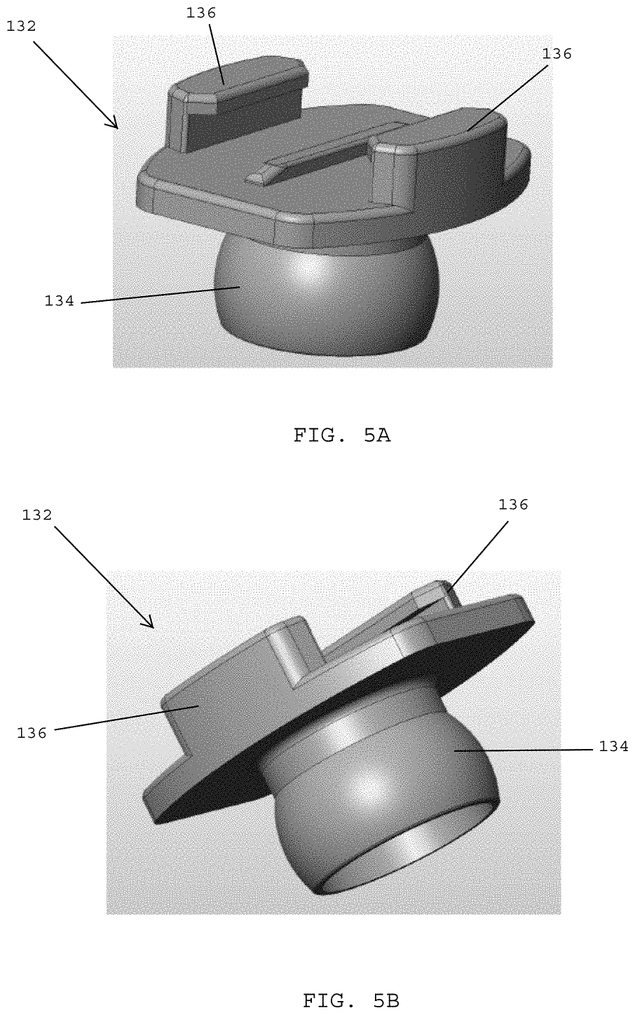

Referring to FIGS. 4A and 4B, in one embodiment, an individual ball and socket segment 108 of a flexible arm 102 (FIG. 1) preferably has a proximal end 142, a distal end 144, and a central opening or conduit that extends from the proximal end 142 to the distal end 144. In one embodiment, the ball and socket segment preferably includes a ball component 148 that is located adjacent the proximal end 142 thereof, a socket component 150 that is preferably located adjacent the distal end 144 thereof, and a reduced diameter waist 152 that is located between the ball component 148 and the socket component 150. In one embodiment, the ball component 148 defines an outer diameter OD₁ and the socket component defines an outer diameter OD₂ that is largely than OD₁. In one embodiment, the ball component 148 of a first ball and socket component may be snap-fit into the socket component 150 of an adjacent ball and socket segment for assembling a flexible arm 102 as shown in FIG. 1.

In one embodiment, the length of a flexible arm may be modified by selectively adding or subtracting the number of ball and socket segments that are snap-fit together. In one embodiment, if an operator wants to lengthen a flexible arm, the operator will snap-fit additional ball and socket segments onto an existing flexible arm. Thereafter, if an operator wants to reduce the length of the flexible arm, the operator may detach one or more of the ball and socket segments from an end of the flexible arm.

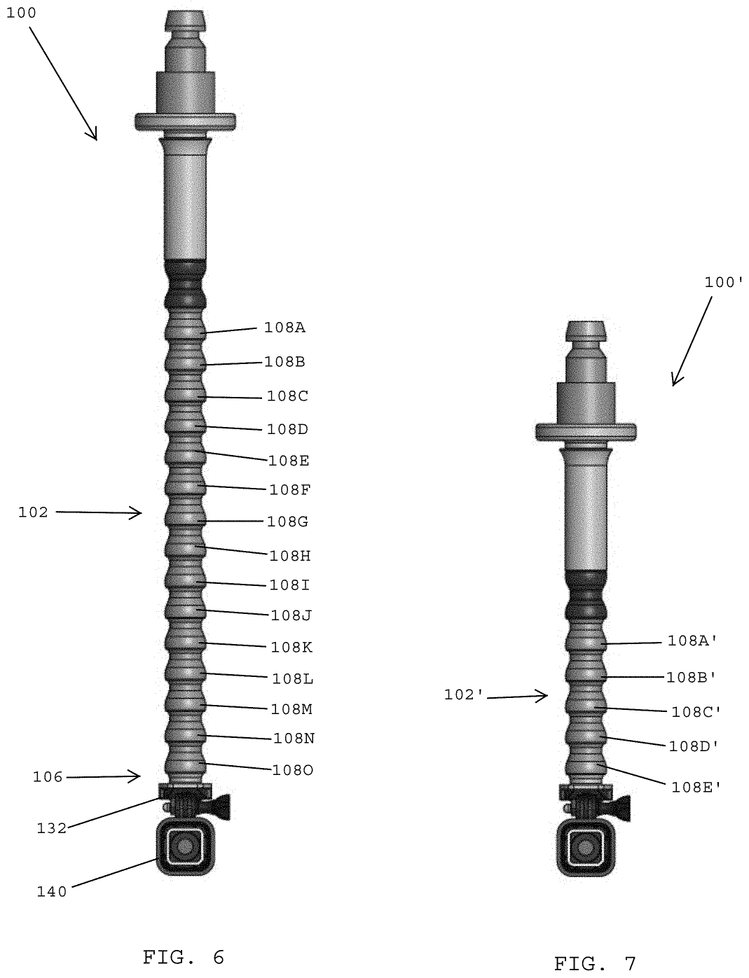

Referring to FIGS. 5A and 5B, in one embodiment, a camera adapter 132 preferably includes a ball component 134 that is adapted to form a snap-fit connection with a socket component 150 of a ball and socket segment 108 (FIG. 4A). The camera adapter 132 preferably includes a camera adapter flange 136 that projects from a distal end of the camera adapter 132 for securing a camera to the camera adapter. After the camera adapter 132 has been snap-fit to an opposing ball and socket segment 108 (FIG. 4A), the camera adapter may pivot and rotate relative to the opposing ball and socket segment.

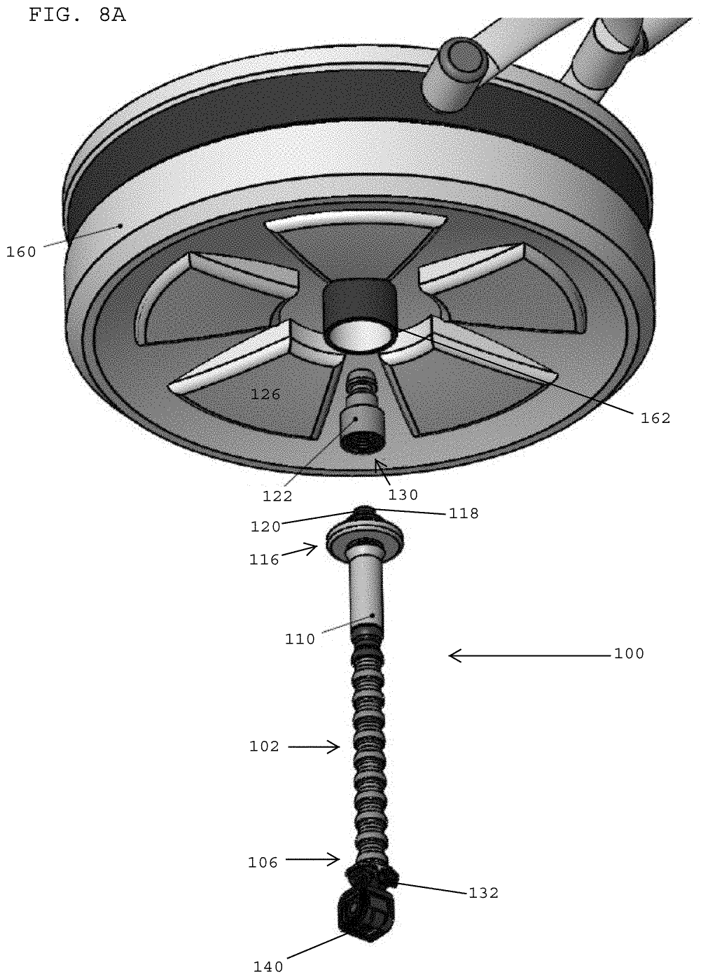

In one embodiment, the length of a flexible arm may be modified by adding additional ball and socket segments to a flexible arm or removing ball and socket segments from an existing flexible arm. Referring to FIG. 6, in one embodiment, a system for securing a camera to an overhead light within a surgical environment preferably has a flexible arm 102 that includes fifteen (15) ball and socket segments 108A-1080 that are snap-fit together to form the flexible arm 102. The camera adapter 132 and the camera 140 are connected with the distal-most end 106 of the flexible arm 102.

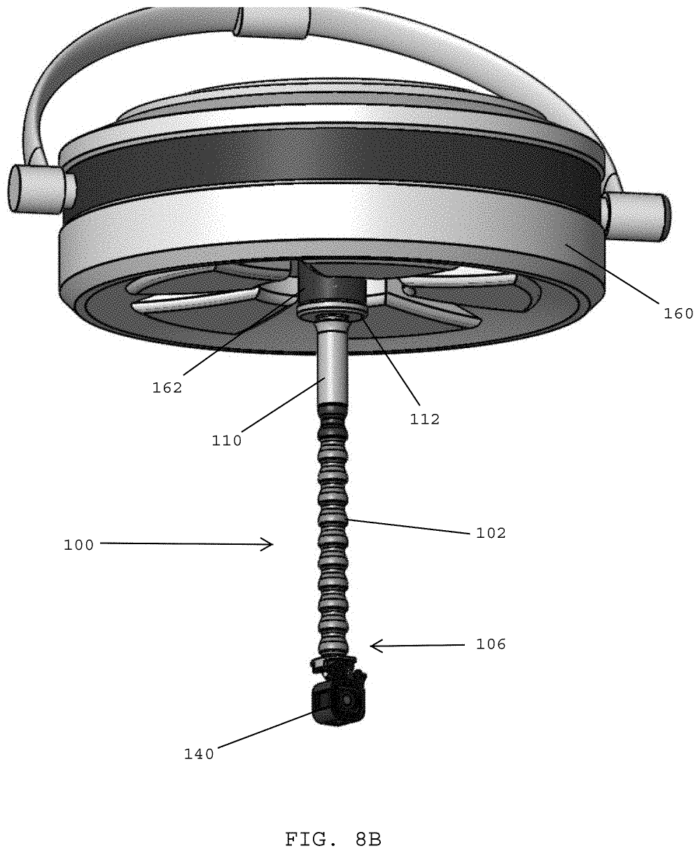

Referring to FIG. 7, in one embodiment, surgical personnel may be required to use a system 100′ having a flexible arm 102′ having a shorter length than the length of the flexible arm 102 shown in FIG. 6. In the embodiment of FIG. 7, the flexible arm 102′ has five (5) ball and socket segments 108A′-108E′ that are snap-fit together to form the flexible arm 102′.

Although the present patent application is not limited by any particular theory of operation, it is believed that providing individual ball and socket segments that may be snap-fit together enables operators to selectively modify the lengths of flexible arms depending upon the needs of medical personnel within a surgical environment. In instances where medical personnel require a longer flexible arm for mounting a camera (e.g., the overhead surgical light is spaced away from an operating room table), the medical personnel may add additional segments to a flexible arm to increase its length. In instances where medical personnel desire a shorter flexible arm (e.g., the overhead surgical light is closer to the operating room table), the segments may be removed from an end of a flexible arm to reduce its overall length. Thus, the ball and socket segments enable the creation of a modular flexible arm that can be selectively modified so that its length matches the needs of medical personnel within a surgical environment.

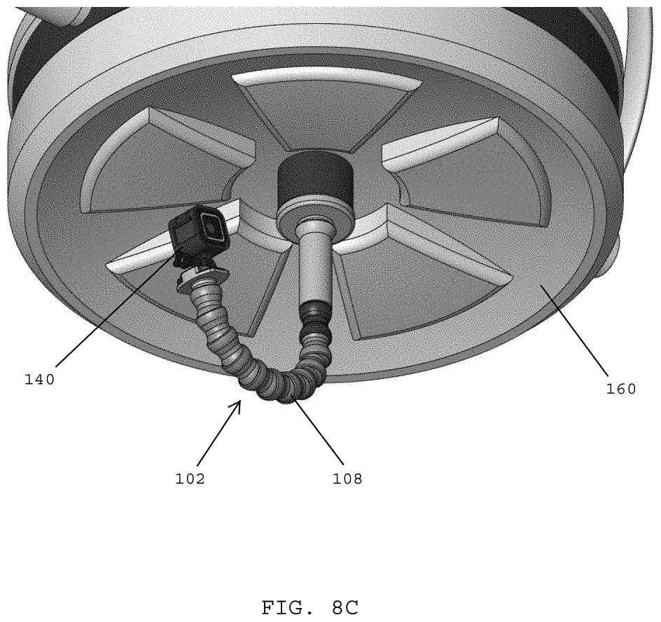

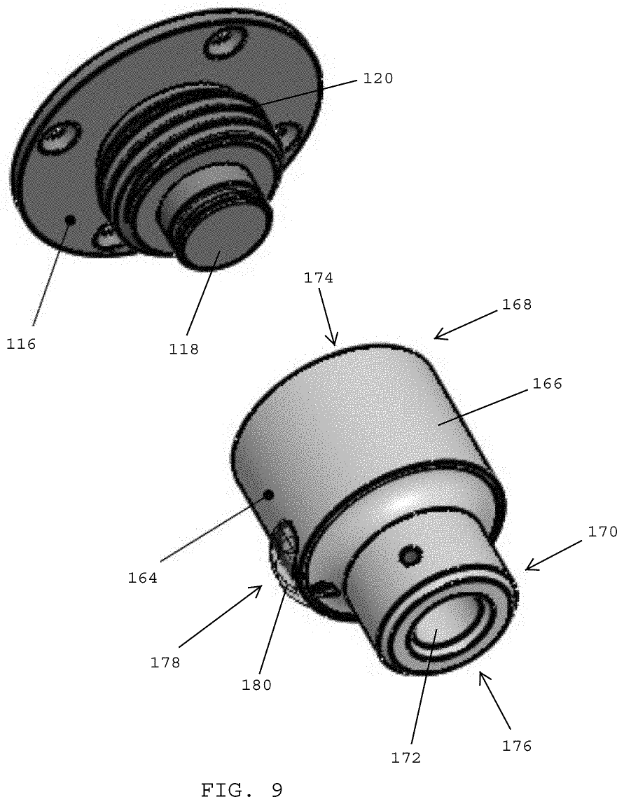

Referring to FIG. 8A, in one embodiment, a system 100 may be used for securing a camera 140 to an overhead light 160. In one embodiment, the overhead light 160 preferably includes a female connector 162 projecting from an underside of the overhead light 160. In one embodiment, the system 100 includes an overhead light connector 124 having the male end 126 that may be inserted into the female connector 162 of the overhead light 160. The external threads 120 of the male projection 118 of the universal adapter 116 may be threaded into the female threads inside the female opening 130 of the overhead light connector 122. In one embodiment, the mounting base 110 projects distally from the universal adapter 120, and the flexible arm 102 extends distally from the mounting base 110. In one embodiment, the camera 140 is mounted to the distal end 106 of the flexible arm 102 via the camera adapter 132.

Referring to FIG. 8B, in one embodiment, after the system 100 has been connected to the overhead light 160, the flexible arm 102 desirably extends away from the female connector 162 at the underside of the overhead light 160. In one embodiment, the mounting base flange 112 of the mounting base 110 is preferably positioned adjacent a lower end of the female connector 162 of the overhead light 160. In one embodiment, the position of the overhead light 160 may be adjusted for fine-tuning the position, location and/or orientation of the camera 140 mounted to the distal end 106 of the flexible arm 102.

Referring to FIG. 8C, in one embodiment, the position, location and/or orientation of the camera 140 may be adjusted by bending the flexible arm 102. As the flexible arm 102 is bent, one or more of the individual ball and socket segments 108 preferably pivot, flex, and/or rotate relative to one another. Thus, the position of the camera 140 may be adjusted by moving the position of the overhead light 160 or pivoting, flexing and/or rotating the flexible arm 102.

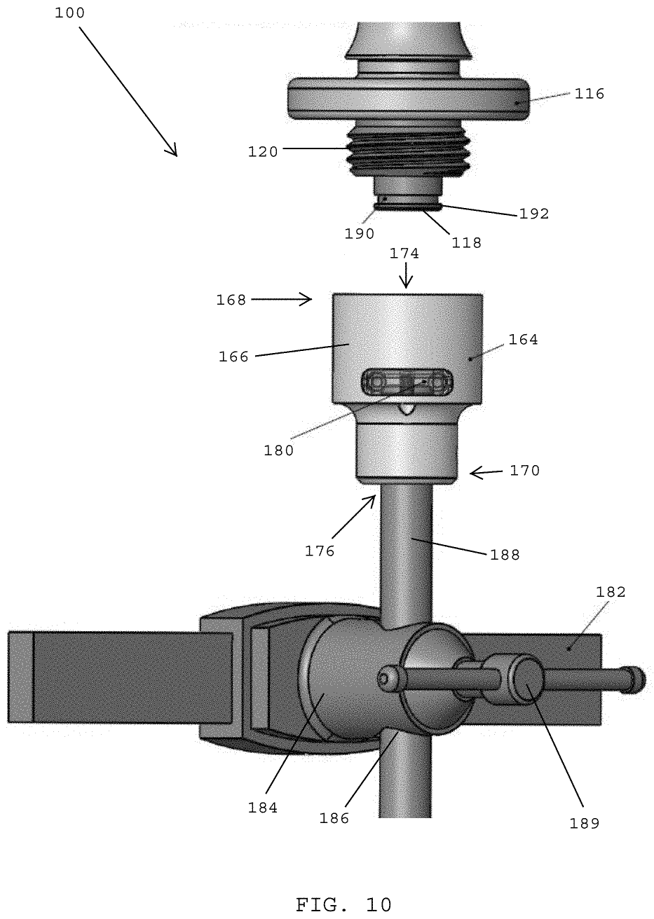

Referring to FIG. 9, in one embodiment, the system disclosed herein may include a post mount adapter 164 that enables the system to be secured to a post rather than an overhead light. In one embodiment, the post mount adapter 164 may include a body 166 having an upper end 168, a lower end 170 and a central conduit 172 that extends from the upper end 168 to the lower end 170. In one embodiment, the post mount adapter 164 desirably has a first female opening 174 at the upper end 168 of the body 166 that is adapted to receive the male projection 118 of the universal adapter 116 (FIG. 1). In one embodiment, the first female opening 174 includes internal threads that are adapted to mesh with the external threads 120 on the male projection 118 of the universal adapter 116.

In one embodiment, the post mount adapter 164 desirably includes a second female opening 176 located at the lower end 170 of the body 166 that is adapted to slide over a post that may be mounted in a surgical environment, such as a post mounted to a table or a bed. In one embodiment, the second female opening 176 has an inner diameter that substantially matches an outer diameter of a post that is located within a surgical environment.

In one embodiment, the post mount adapter 164 preferably includes a locking assembly 178. In one embodiment the locking assembly 178 may include a depressible button 180 that is connected with a locking ring. In one embodiment, the locking ring is disposed inside the first female opening 174 of the body 166, and the locking ring is urged into a locked position by a spring. In one embodiment, the locking ring 180 is preferably located between the upper end 168 of the body 166 and the upper end of the internal threads found inside the post mount adapter 164.

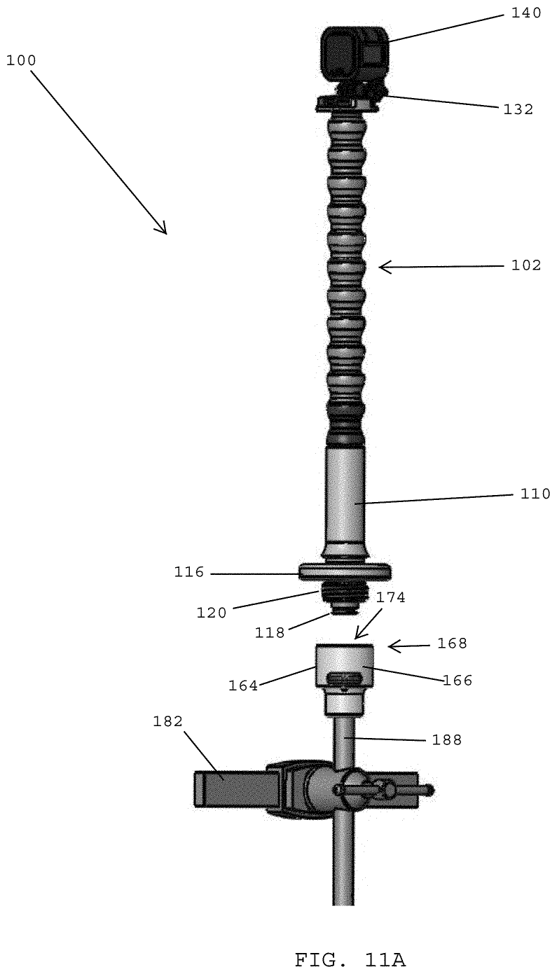

Referring to FIG. 10, in one embodiment, a securing assembly 182 preferably includes a post mount 184 having an opening 186 that is adapted to receive a post 188, such as a vertically extending post. In one embodiment, the securing assembly 182 preferably includes a clamp 189 may be loosened for enabling the post 188 to slide up and down within the post mount opening 186. In one embodiment, when the post 188 has been positioned at a desired elevation relative to the securing assembly 182, the clamp 189 may be tightened for locking the post 188 in place.

In one embodiment, the post mount adapter 164 (FIG. 9) is positioned atop the post 188. In one embodiment, the second female opening 176 at the lower end 170 of the body 166 of the post mount adapter slides over the upper end of the post 188 for positioning the body 166 on the post 188. In order to secure the universal adapter 116 (FIG. 9) to the post 188, the male projection 118 is preferably aligned with the first female opening 174 at the upper end 168 of the body 166. In one embodiment, the male projection 118 desirably includes an annular depression 190 that defines a locking flange 192 at the end of the male projection 118. In one embodiment, the locking ring disposed inside the body 166 of the post mount adapter 164 is adapted to sit within the annular groove 190 and engage the locking flange 192 for locking the male projection 118 within the first female opening 174 of the body 166. In one embodiment, after the universal adapter 116 has been secured to the post mount adapter 164, the universal adapter may only be decoupled (e.g., unthreaded) from the body 166 of the post mount adapter 164 by depressing the release button 180 for uncoupling the locking ring from the locking flange 192 of the male projection 118, whereupon the universal adapter 116 may be unthreaded from the post mount adapter 164.

In one embodiment, in order to secure the universal adapter 116 to the post mount adapter 164, the external threads 120 on the male projection 118 are inserted into the first female opening 174 of the post mount adapter 164 and threaded into the internal threads of the body 166. As the external threads 120 of the universal adapter 116 are threaded into the female threads of the body 166, the locking ring of the post mount adapter 164 eventually engages the annular depression 190 formed in the male projection 118 for locking the male projection in place within the body 166 of the post mount adapter 164. After the locking ring locks the universal adapter to the post mount adapter, the universal adapter may only be decoupled from the post mount adapter by engaging the release button to move the locking ring into an unlocked position.

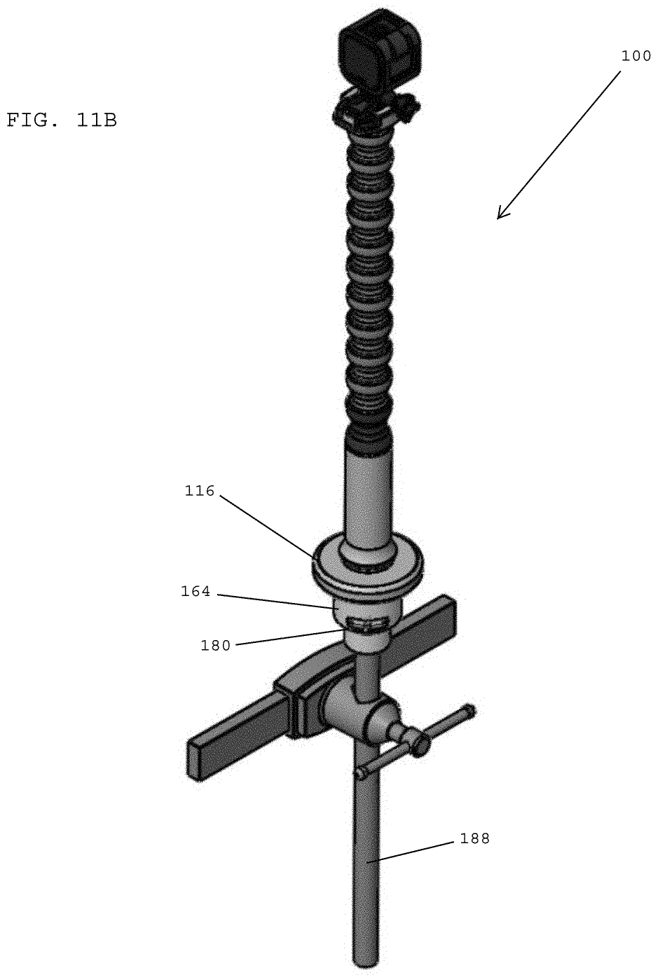

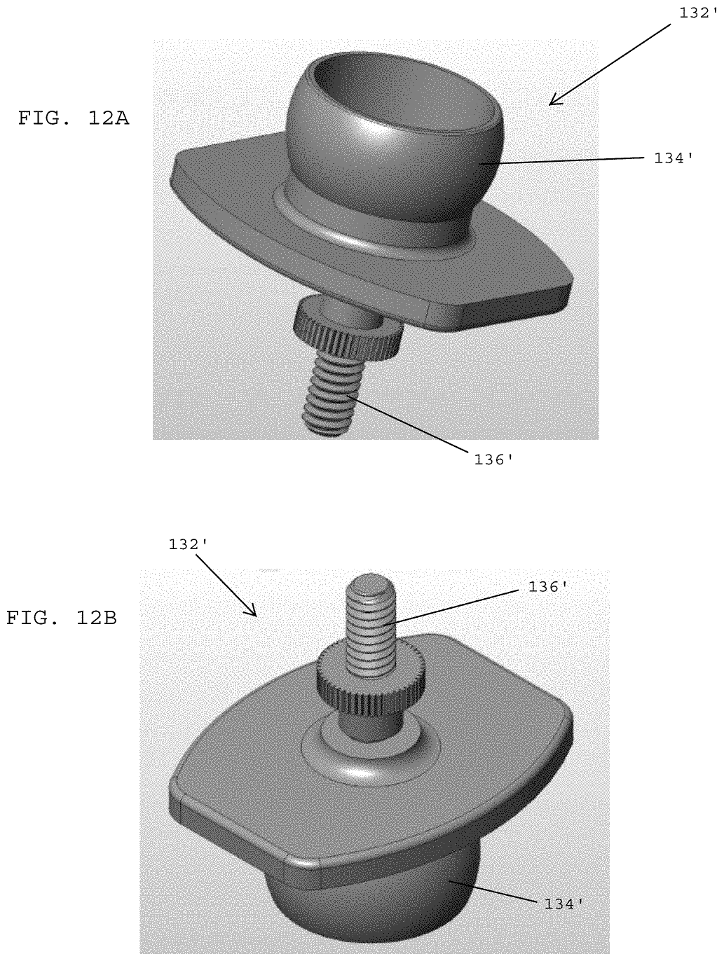

Referring to FIG. 11A, in one embodiment, a system 100 for securing a camera 140 within a surgical environment preferably includes the universal adapter 116, the mounting base 110, the flexible arm 102 and the camera adapter 132 that is connected with the camera 140. In one embodiment, after the post mount adapter 164 has been secured atop the post 188, the male projection 118 of the universal adapter 116 may be inserted into the first female opening 174 at the upper end 168 of the body 166 of the post mount adapter 164. The external threads 120 on the male projection 118 preferably mesh with internal threads located inside the body 166 of the post mount adapter 164 for threading the universal adapter 116 into the post mount adapter 164.

Referring to FIG. 11B, in one embodiment, the universal adapter 116 is secured to the post amount adapter 164 to connect the camera system 100 to the post 188. The universal adapter 116 will remain secured to the post mount adapter 164 so long as the internal locking ring of the post mount adapter 164 remains engaged with the annular depression 190 and the locking flange 192 on the male projection 118 of the universal adapter 116 (FIG. 10). In one embodiment, in order to detach the universal adapter 116 from the post mount adapter 164, the depressible button 180 may be depressed for shifting the locking ring of the post mount adapter 164 out of the annular depression 190 (FIG. 10) of the male projection of the universal adapter 116, whereupon the external threads on the male projection of the universal adapter 116 may be unthreaded from the female threads of the post mount adapter 164 to detach the system 100 from its connection with the post 188. The locking assembly including the locking ring of the post mount adapter preferably prevents inadvertent or accidental uncoupling of the system from the post 188

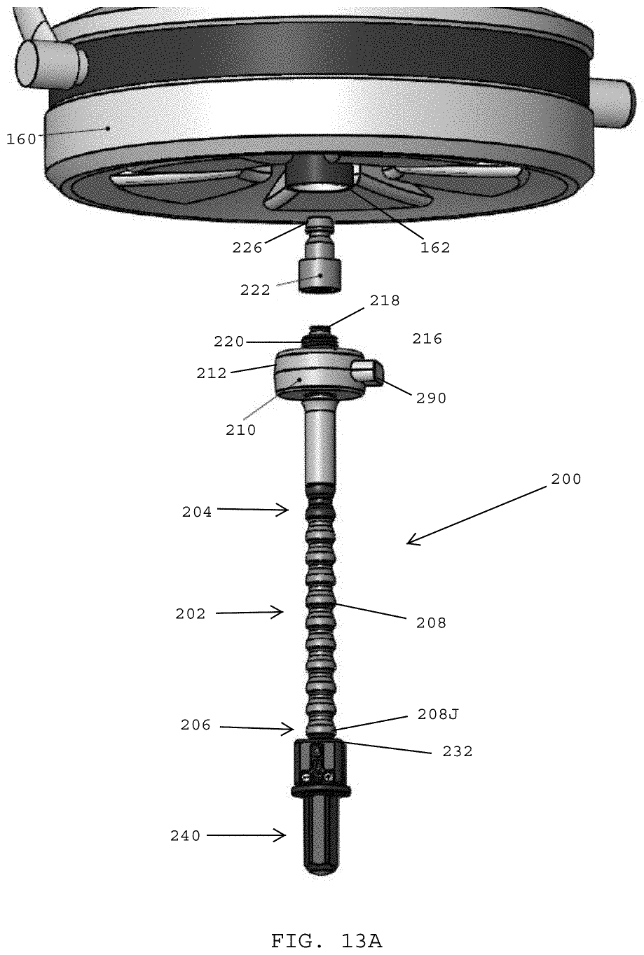

In one embodiment, different types of camera adapters may be utilized with the system for securing different types of cameras to the flexible arm of the system disclosed herein. Referring to FIGS. 12A and 12B, in one embodiment, a camera adapter 132′ desirably includes a ball component 134′ that is adapted to form a snap-fit connection with a socket component of an adjacent ball and socket segment (FIG. 1). In one embodiment, the camera adapter 132′ desirably includes an externally threaded post 136′ that is adapted to be threaded into an internally threaded opening found on a camera. In one embodiment, after the threaded post 136′ is threaded into the internally threaded opening of a camera, the ball component 134′ of the camera adapter 132′ is snap-fit into a socket component of a ball and socket segment disclosed herein for being coupled with an end of a flexible arm 102 (FIG. 1).

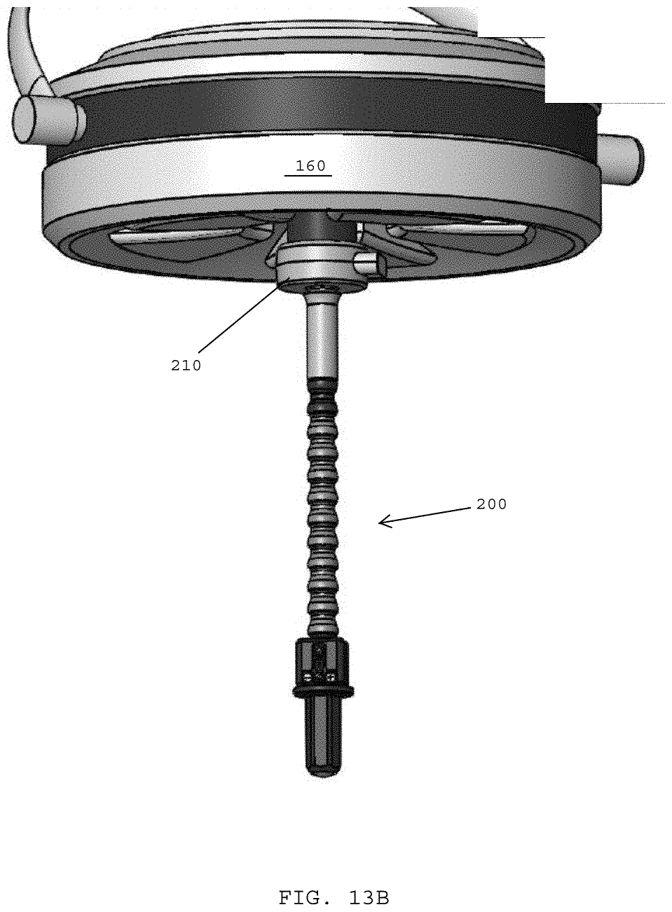

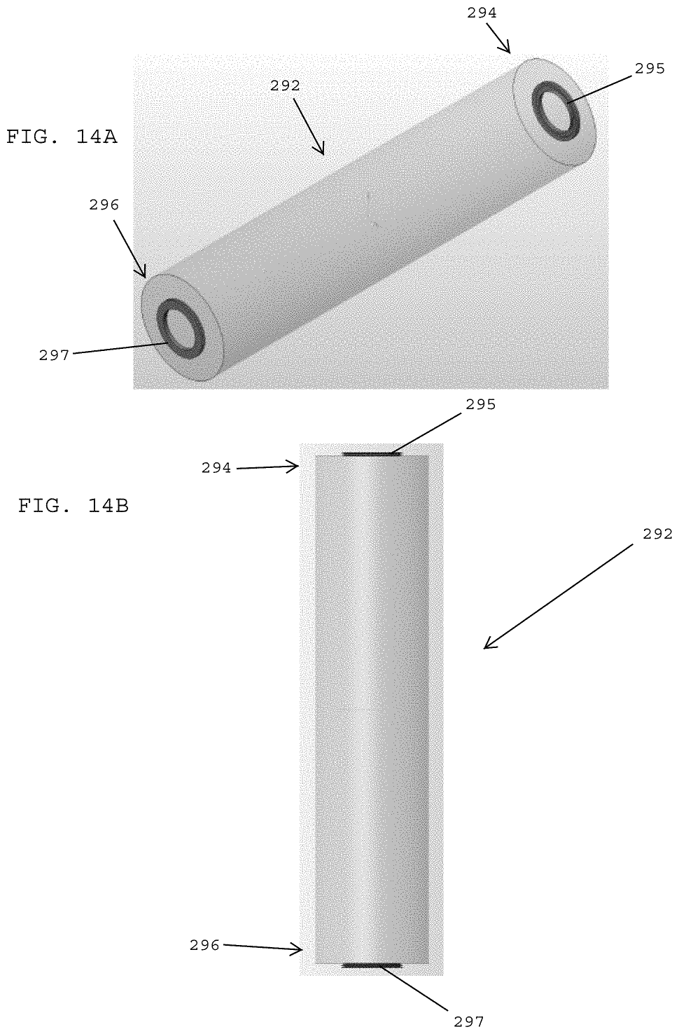

Referring to FIG. 13A, in one embodiment, a system 200 for providing and securing supplemental lighting within a surgical environment preferably includes a flexible arm 202 having a proximal end 204 and a distal end 206. The flexible arm 202 desirably includes a plurality of ball and socket segments 208 that may be snap-fit together. In one embodiment, the ball and socket segments 208 are adapted to pivot relative to one another for enabling the flexible arm 202 to flex and bend. In one embodiment, the system for securing supplemental lighting desirably includes a mounting base 210 having a mounting base flange 212, which is connected with a universal adapter 216 having a male projection 218 with external threads 220. The system desirably includes an overhead light connector 222 that may be inserted into a female opening 162 of an overhead light 160. In one embodiment, the external threads 220 on the male projection 218 are preferably inserted into a female opening at the lower end of the overhead light connector 222 for securing the system 200 to the overhead light connector 222, which, in turn, is inserted into the female opening 162 of the overhead light 160.

In one embodiment, the system 200 for providing supplemental lighting desirably includes a light fixture 240 that is coupled with the distal end 206 of the flexible arm 202 via a light fixture adapter 232. The light fixture adapter 232 preferably includes a ball component that forms a snap-fit connection with a socket component of a distal-most ball and socket segment 208J of the flexible arm 202.

In one embodiment, the system 200 preferably includes a power source for providing power to the light fixture 240. In one embodiment, the power source may include a rechargeable battery 290 that may be inserted into the body of the mounting base 210. One or more electrically conductive wires may extend through the ball and socket segments of the flexible arm 202 for transmitting power from the power source 290 to the light fixture 240. In one embodiment, the light fixture 240 desirably includes one or more lights, such as LED lights, that provide supplemental lighting that is in addition to the lighting provided by the overhead light 160. In one embodiment, the flexible arm 202 may be flexed, bent and/or rotated for configuring the light fixture 240 at a desired angle and/or orientation relative to a surgical environment. In one embodiment, the length of the flexible arm 202 may be modified by adding additional ball and socket segments 208 to the flexible arm for lengthening the flexible arm, or removing ball and socket segments from the flexible arm to shorten the flexible arm.

Referring to FIGS. 13A and 13B, in one embodiment, after the external threads 220 of the male projection 218 of the mounting base 210 have been threaded into the female threads of the overhead light connector 220, the male projection 226 at the upper end of the overhead light connector is desirably inserted into the female opening 162 of the overhead light 160 for securing the supplemental lighting system 200 to the overhead light 160.

Referring to FIGS. 14A and 14B, in one embodiment, a system for securing cameras and supplemental lighting within a surgical environment may include a sanitary sleeve 292 having a first end 294 with a first band 295 and a second end 296 with a second band 297. The sanitary sleeve 292 is preferably positioned over one or more of the components of the system disclosed herein for maintaining a sterile environment within an operating room. In one embodiment, the first and second bands 295, 297 may be elastic bands.

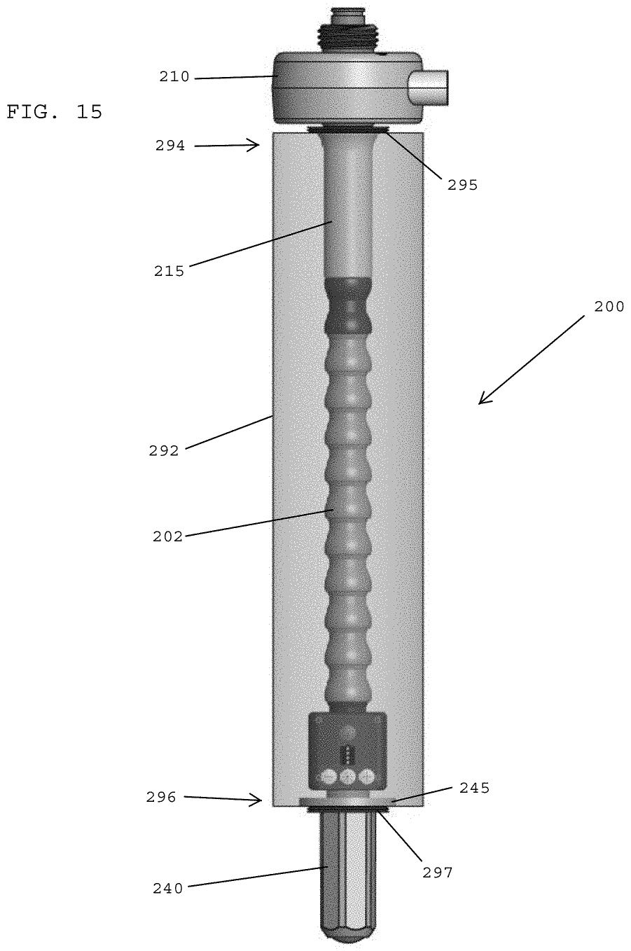

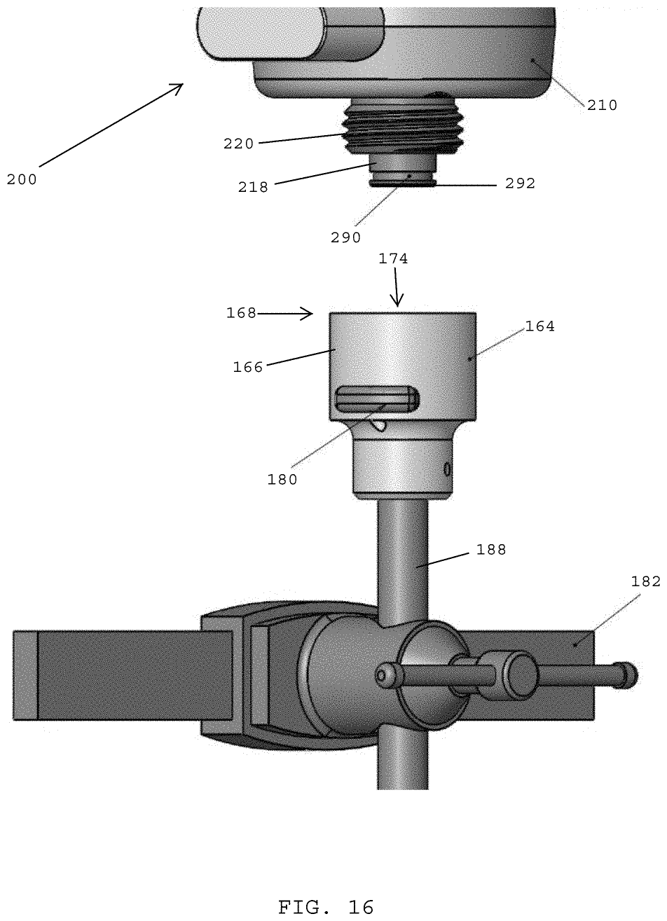

Referring to FIG. 15, in one embodiment, the sanitary sleeve 292 may be positioned over the handle 215 of a mounting base 210, the flexible arm 202 and an upper portion of the light fixture 240. In one embodiment, the first elastic band 295 at the upper end 294 of the sanitary sleeve 292 is secured over the proximal end of the handle 215 of the mounting base 210. The second elastic band 297 at the lower end 296 of the sanitary sleeve 292 may be secured over a mounting flange 245 provided on the light fixture 240. The sanitary sleeve 292 desirably enables surgical personnel to engage components of the system and flex and bend the flexible arm 202 of the supplemental light system 200 by grasping the outer surface of the sanitary sleeve 292 so as to maintain a sterile environment within an operating room. In one embodiment, the sanitary sleeve 202 is positioned over the system by passing the first elastic band 295 over the flange 245 of the light fixture 240 and sliding the sanitary sleeve 292 toward the mounting base 210 until the first elastic band 295 is positioned over the proximal end of the handle 215 as shown in FIG. 15. The second elastic band 297 may then be stretched for passing over the distal end of the light fixture 240 until the second elastic band 297 abuts against the camera flange 245.

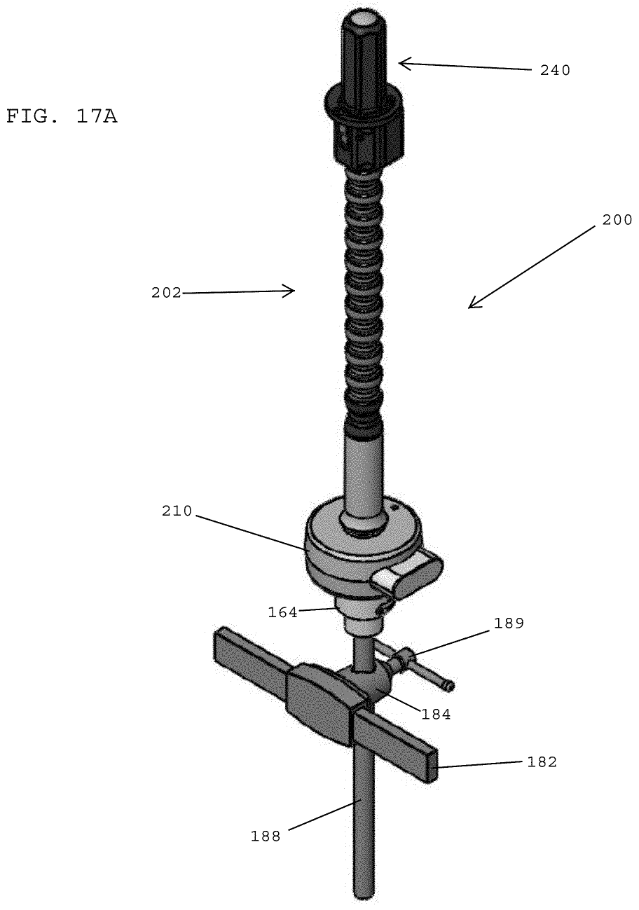

Referring to FIG. 16, the supplemental lighting system 200 shown and described herein may be secured atop a post 188 of a securing assembly 182. In one embodiment, the post mount adapter 165 (FIG. 10) disclosed herein is secured to an upper end of the post 188. The post mount adapter 165 preferably includes the depressible button 180 that is connected to an internal locking ring disposed inside the body 166 of the post mounting adapter. In one embodiment, the mounting base 210 of the system 200 preferably includes a male projection 218 having an annular depression 290 and a locking flange 292 defined by the annular depression 290. When the male projection 218 is inserted into the first female opening 174 at the upper end 168 of the body 166 of the post mount adapter 164, the locking flange 292 on the male projection 218 preferably engages the locking ring disposed inside the body 166. In one embodiment, after the external threads 220 of the male projection 218 have been threaded into the internal threads of the body 166 of the post mount adapter 164, the external threads 220 may only be unthreaded from the internal threads of the post mount adapter 164 after the depressible button 180 has been depressed for releasing the internal locking ring from the locking flange 292 of the male projection 218.

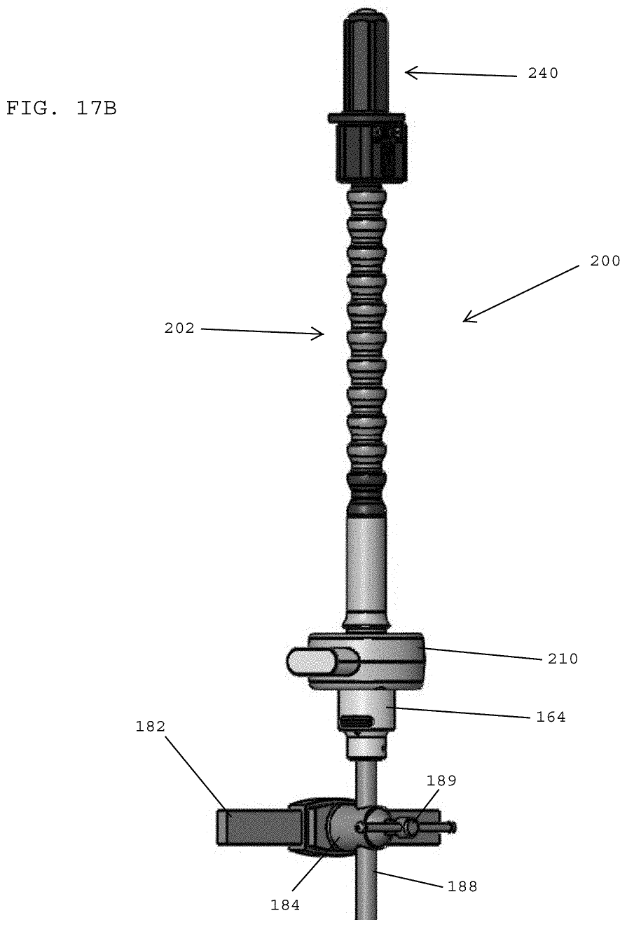

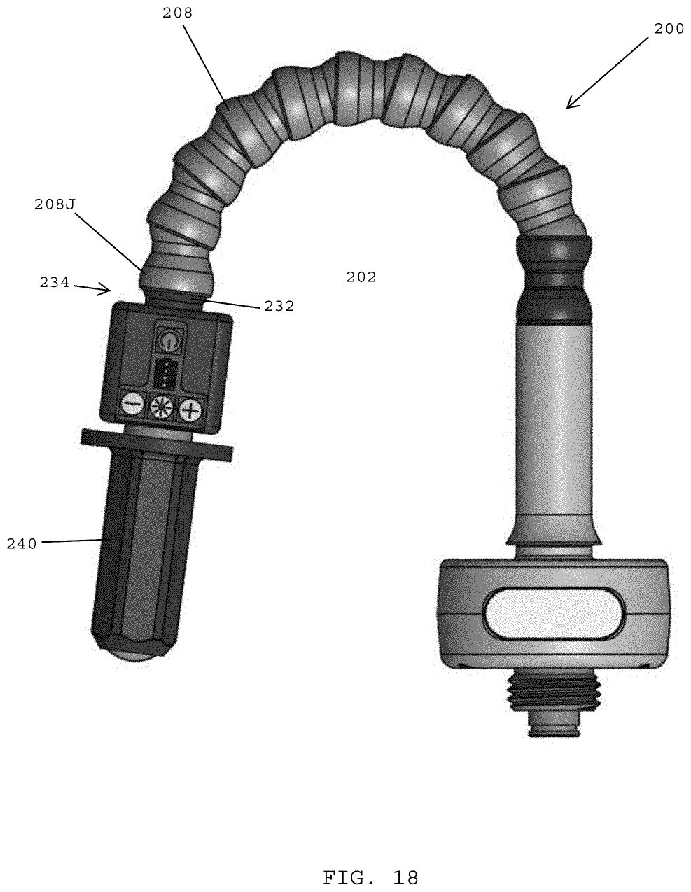

Referring to FIGS. 17A and 17B, after the mounting base 210 has been secured to the post mount adapter 164, which, in turn, is connected with a post 188, the supplemental lighting system 200 preferably extends above an upper end of the post 188 of the securing assembly 182. The post 188 may be vertically adjusted by loosening the clamp 189 which enables the post 188 to slide up and down within the post mount opening 186 of the post mount 188. The position of the light fixture 240 may be adjusted by bending, flexing, and/or rotating the flexible arm 202 to direct and/or orient the light generated by the light fixture 240 at a desired location within a surgical environment.

Referring to FIG. 18, in one embodiment, the flexible arm 202 of the system 200 desirably includes ball and socket segments 208 that are snap-fit together. The systems 200 preferably includes a light fixture adapter 232 having a ball component 234 that forms a snap-fit connection with a socket component of a distal-most ball and socket segment 208J of the flexible arm 202. The flexible arm 202 may be bent, flexed, and/or rotated to a desired configuration or orientation to direct the light generated by the light fixture 240 toward a desired location within a surgical environment. The ball and socket segments 208 preferably hold the flexible arm 202 in the bent configuration selected by surgical personnel, until the surgical personnel desire to change the configuration and/or orientation of the light fixture 240.

While the foregoing is directed to embodiments of the present invention, other and further embodiments of the invention may be devised without departing from the basic scope thereof, which is only limited by the scope of the claims that follow. For example, the present invention contemplates that any of the features shown in any of the embodiments described herein, or incorporated by reference herein, may be incorporated with any of the features shown in any of the other embodiments described herein, or incorporated by reference herein, and still fall within the scope of the present invention.

What is claimed is:

1. A system for use during surgical procedures comprising:
a flexible arm having a first end and a second end;
said flexible arm including a plurality of ball and socket segments that are interconnected, wherein adjacent ones of said interconnected ball and socket segments are configured to pivot relative to one another;
a universal adapter coupled with the first end of said flexible arm; and
a component adapter coupled with the second end of said flexible arm, wherein said component adapter comprises a ball component that forms a snap-fit connection with one of said ball and socket segments.

2. The system as claimed in claim 1, further comprising a camera secured to said component adapter, wherein said interconnected ball and socket segments enable the second end of said flexible arm to flex relative to the first end of said flexible arm for selectively modifying the orientation of said camera.

3. The system as claimed in claim 1, further comprising a light fixture secured to said component adapter, wherein said interconnected ball and socket segments enable the second end of said flexible arm to flex relative to the first end of said flexible arm for selectively modifying the orientation of said light fixture.

4. The system as claimed in claim 1, wherein said universal adapter coupled with the first end of said flexible arm comprises:
a male projection having a free end;
external threads provided on an outer surface of said male projection;
a locking flange provided at the free end of said male projection; and
an annular groove formed in the outer surface of said male projection that is located between said locking flange and said external threads.

5. The system as claimed in claim 4, further comprising:
an overhead light connector having a female opening with internal threads;
wherein said external threads of said male projection are threaded into said internal threads of said female opening of said overhead light connector for securing said universal adapter to said overhead light adapter.

6. The system as claimed in claim 5, wherein said overhead light connector has an end coupled with an overhead light for securing said universal adapter and said flexible arm to said overhead light.

7. The system as claimed in claim 6, wherein said overhead light connector comprises a locking assembly that is moveable between a locked position that enables said universal adapter to be unthreaded from said overhead light connector and a locked position that prevents said universal adapter from being unthreaded from said overhead light connector.

8. The system as claimed in claim 7, wherein said locking assembly comprises a locking ring that is seated in said annular groove of said male projection when said locking assembly is in the locked position, and that is spaced from said annular groove of said male projection when said locking assembly is in the unlocked position.

9. The system as claimed in claim 4, further comprising:
a post mount adapter including a body having a first end, a second end, and a central conduit extending from the first end to the second end;
a first female opening located at the first end of said body of said post mount adapter that is adapted to receive the male projection of said universal adapter, said first female opening including internal threads that are adapted to mesh with the external threads of said male projection of said universal adapter;
a second female opening located at the second end of said body of said post mount adapter that is adapted to slide over a post.

10. The system as claimed in claim 9, further comprising a post having an end disposed within said second female opening of said post mount adapter for securing said post mount adapter to said post.

11. The system as claimed in claim 10, said post mount adapter further comprising a locking assembly that is moveable between a locked position that enables said universal adapter to be unthreaded from said post mount adapter and a locked position that prevents said universal adapter from being unthreaded from said post mount adapter.

12. The system as claimed in claim 11, wherein said locking assembly of said post mount adapter includes a locking ring that is seated in an annular groove of said male projection when said locking assembly is in the locked position, and that is spaced from said annular groove of said locking assembly when said locking assembly is in the unlocked position.

13. The system as claimed in claim 1, further comprising a mounting base having a first end that is connected with said universal adapter, a second end having a socket component that forms a snap-fit connection with one of said ball and socket segments of said flexible arm, and an elongated handle that extends between the first end and the second end of said mounting base.

14. A system for use during surgical procedures comprising:
a flexible arm having a first end and a second end, said flexible arm including a plurality of ball and socket segments that are interconnected, wherein adjacent ones of said interconnected ball and socket segments are configured to pivot relative to one another;
a universal adapter coupled with the first end of said flexible arm, said universal adapter including a male projection having a free end and an outer surface that extends to the free end, external threads provided on the outer surface of said male projection, a locking flange provided at the free end of said male projection, and an annular groove formed in the outer surface of said male projection that is located between said locking flange and said external threads;
an overhead light connector having a female opening with internal threads, wherein said external threads of said male projection of said universal adapter are threaded into said internal threads of said female opening of said overhead light connector for securing said universal adapter and said flexible arm to said overhead light connector, and wherein said overhead light connector comprises a locking assembly that is moveable between a locked position that enables said universal adapter to be unthreaded from said overhead light connector and a locked position that prevents said universal adapter from being unthreaded from said overhead light connector;
a camera adapter coupled with the second end of said flexible arm, wherein said camera adapter comprises a ball component that forms a snap-fit connection with one of said ball and socket segments; and
a camera secured to said camera adapter, wherein said interconnected ball and socket segments enable the second end of said flexible arm to flex relative to the first end of said flexible arm for selectively modifying the orientation of said camera.

16. The system as claimed in claim 15, wherein said camera is selected from the group consisting of electronic cameras, digital cameras, video cameras, still cameras, and film cameras.

17. The system as claimed in claim 15, wherein said camera is in wireless communication with a central processing unit for transmitting images to said central processing unit.

18. A kit for recording surgical procedures and providing supplemental lighting in a surgical environment comprising:
a plurality of ball and socket segments that are configured to be interconnected for forming a flexible arm, each said ball and socket segment including a ball component at a first end and a socket component at a second end, wherein one or more of said ball and socket segments may be added to said flexible arm for increasing a length of said flexible arm or removed from said flexible arm for reducing the length of said flexible arm;
a universal adapter securable to at least one of said ball and socket segments;
a component adapter having a ball component configured to form a snap-fit connection with at least one of said ball and socket segments;
a camera securable to said component adapter;
a light fixture securable to said component adapter;
an overhead light connector having a female opening with internal threads, wherein said universal adapter has external threads on a male projection thereof that are adapted to be threaded into said internal threads of said female opening of said overhead light connector for securing said universal adapter to said overhead light adapter;
a post mount adapter including a body having a first end, a second end, and a central conduit extending from the first end to the second end, a first female opening located at the first end of said body of said post mount adapter that is adapted to receive the male projection of said universal adapter, said first female opening including internal threads that are adapted to mesh with the external threads of said male projection of said universal adapter, and a second female opening located at the second end of said body of said post mount adapter that is adapted to slide over a post.

19. The system as claimed in claim 18, wherein said overhead light connector comprises a locking assembly that is moveable between a locked position that enables said universal adapter to be unthreaded from said overhead light connector and a locked position that prevents said universal adapter from being unthreaded from said overhead light connector.

20. The system as claimed in claim 18, wherein said post mount adapted comprises a locking assembly that is moveable between a locked position that enables said universal adapter to be unthreaded from said post mount adapter and a locked position that prevents said universal adapter from being unthreaded from said post mount adapter.
